(19) United States
(12) Patent Application Publication
(10) Pub. No.: US 2025/0041539 A1
(43) Pub. Date: Feb. 6, 2025

(54) NEBULIZER SYSTEM

(71) Applicant: OMRON HEALTHCARE Co., Ltd., Muko-shi (JP)

(72) Inventors: Hidetaka TOGO, Kyoto (JP); Hiroko YOSHINO, Kyoto (JP)

(21) Appl. No.: 18/924,010

(22) Filed: Oct. 23, 2024

Related U.S. Application Data
(63) Continuation of application No. PCT/JP2023/003600, filed on Feb. 3, 2023.

(30) Foreign Application Priority Data
Jul. 21, 2022 (JP) 2022-116661

(51) Int. Cl. A61M 11/00

(52) U.S. Cl. CPC A61M 11/005

(57) ABSTRACT

A nebulizer system of the present invention includes a computer device and a nebulizer head directly connected to the computer device by a USB cable. The computer device supplies a DC voltage through a Vbus line and a GND line of the USB cable, and supplies a control signal having a square waveform through a D+ line and a D− line of the USB cable. The nebulizer head includes a first resonance circuit including a first coil and a first capacitor, a conversion circuit that applies a DC voltage intermittently or alternately polarity-reversedly according to a control signal of the first resonance circuit to generate an AC voltage having a sinusoidal waveform, and an atomizer that is driven by the AC voltage having the sinusoidal waveform and atomizes and ejects a liquid.

### CROSS-REFERENCE TO RELATED APPLICATIONS

This is a continuation application of International Application No. PCT/JP2023/003600, with an International filing date of Feb. 3, 2023, which claims priority of Japanese Patent Application No. 2022-116661 filed on Jul. 21, 2022, the entire content of which is hereby incorporated by reference.

### TECHNICAL FIELD

The present invention relates to a nebulizer system, and more specifically to a nebulizer system including a computer device capable of supplying a USB signal and a nebulizer head connected to the computer device by a USB cable.

### BACKGROUND ART

Conventionally, as this type of nebulizer system, for example, as disclosed in Patent Document 1 (Japanese Utility Model Registration No. 3199314), a nebulizer system including a computer device capable of supplying a USB signal, a controller connected to the computer device by a USB cable, and a mesh nebulizer head connected to the controller by a proprietary cable is known. The controller includes a housing, and a booster circuit including a DC-DC converter, a microcontroller (processor), and a drive circuit in which a high-speed MOSFET driver is incorporated are mounted on the housing. The booster circuit generates a DC voltage of nominal 12 V from a DC of 5V (exactly 4.75 V to 5.25 V) included in the USB signal from the computer device. Using the output of the booster circuit, the microcontroller generates a square waveform (pulse-width modulated signal) of 120 to 150 KHz and sends the square waveform to the drive circuit. The drive circuit generates an AC voltage (sinusoidal waveform of approximately 100 V) from the square waveform of 120 to 150 KHz using a series inductor. The nebulizer head has a piezoceramic element, receives liquid from a supply container, and is driven by the 120 to 150 KHz sinusoidal waveform from the drive circuit to generate aerosol.

### SUMMARY OF THE INVENTION

The nebulizer system as described above is desirably configured compactly with a small number of components from the viewpoint of being portable. However, in the system of Patent Document 1, a controller (including a processor) is interposed between a computer device capable of supplying a USB signal and a nebulizer head. For this reason, there is a problem that the number of components increases and that the components are bulky, for a nebulizer system.

Therefore, an object of the present invention is to provide a nebulizer system that can be configured compactly with a small number of components.

In order to achieve the object, a nebulizer system of the present disclosure is a nebulizer system for atomizing and ejecting a liquid, the nebulizer system comprising:
a computer device configured to supply a signal according to a USB standard through a USB cable,
wherein the computer device supplies a DC voltage through a Vbus line and a GND line of the USB cable, and supplies a control signal having a square waveform through a D+ line and a D− line of the USB cable; and
a nebulizer head directly connected to the computer device by the USB cable,
wherein the nebulizer head includes:
a first resonance circuit including a first coil and a first capacitor,
a conversion circuit that applies the DC voltage intermittently or alternately polarity-reversedly according to the control signal to the first resonance circuit to generate an AC voltage having a sinusoidal waveform, and
an atomizer that is driven by the AC voltage having the sinusoidal waveform and atomizes and ejects the liquid.

In the present specification, the “USB cable” is intended to include a cable capable of transmitting a signal according to the USB standard and a connector (this is referred to as a “USB connector”) provided at an end portion of the cable.

The “control signal” refers to a D+ signal and a D− signal, which are differential signals.

The “directly connected” by the USB cable means that a connection is made between the computer device and the nebulizer head without any intervening of a controller (including a processor) in the conventional example. It should be noted that it is not excluded that the computer device and/or the nebulizer head comprise(s) a USB connector, to which the USB connector at the end portion of the USB cable can be connected.

### BRIEF DESCRIPTION OF DRAWINGS

FIG. 1 is a view showing a schematic configuration of a nebulizer system according to an embodiment of the present invention.

FIG. 2 is a block diagram of a control system of a smartphone constituting the nebulizer system.

FIG. 3 is a view showing a nebulizer head constituting the nebulizer system as viewed obliquely in a disassembled state.

FIG. 4 is a view schematically showing an internal structure of the nebulizer head assembled as viewed from a side direction.

FIG. 5 is a diagram illustrating an entire electric circuit included in the nebulizer head.

FIG. 6 is a diagram showing a modification of the electric circuit in FIG. 5.

FIGS. 7A, 7B, and 7C are diagrams showing a control flow by a control unit included in the smartphone.

FIG. 8 is a view showing a relationship between a control signal having a square waveform output from the smartphone to the nebulizer head and an AC voltage having a sinusoidal waveform created using the control signal in the nebulizer head.

FIG. 9 is a view showing a state in which a frequency of the AC voltage having the sinusoidal waveform in FIG. 8 is changed.

FIG. 10 is a view showing a usage mode in which a user uses the nebulizer system.

### DETAILED DESCRIPTION

Hereinafter, embodiments of the present invention will be described in detail with reference to the drawings.

### (Schematic Configuration of Nebulizer System)

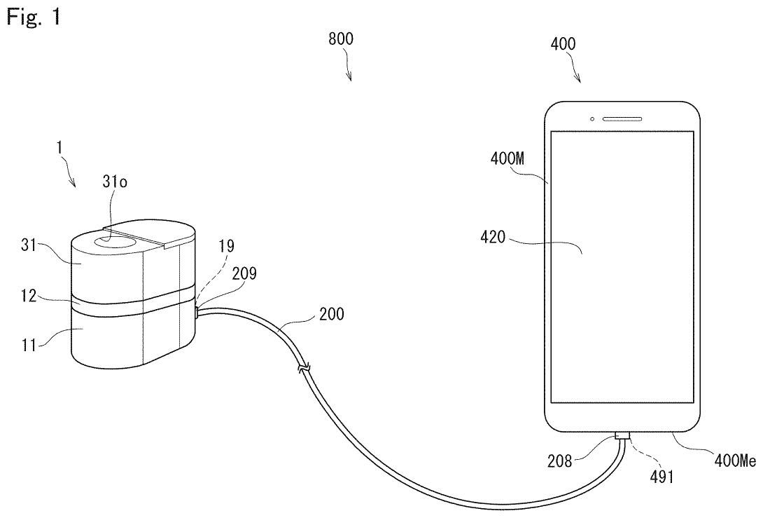

FIG. 1 shows a schematic configuration of a nebulizer system 800 according to an embodiment of the present invention. The nebulizer system 800 is a nebulizer system for atomizing and ejecting a liquid, and includes a smartphone 400 as a computer device and a nebulizer head 1 directly connected to the smartphone 400 by a USB cable 200. In this example, the USB cable 200 has male USB connectors 208 and 209 at both ends, respectively. The USB connector 208 is detachably connected to a female USB connector 491 provided in the smartphone 400, and the USB connector 209 is detachably connected to a female USB connector 19 provided in the nebulizer head 1.

### (Configuration of Smartphone)

The smartphone 400 is configured to cause the nebulizer head 1 to perform a nebulizing operation described below by installing nebulizer application software (computer program) in a general commercially available smartphone.

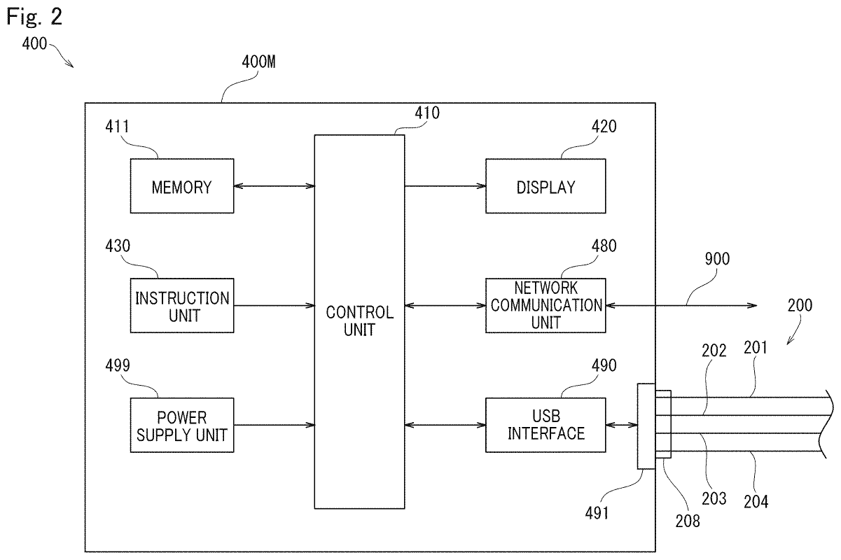

Specifically, as shown in FIG. 2, the smartphone 400 includes a main body 400M, and further includes a control unit 410, a memory 411, a display 420, an instruction unit 430, a network communication unit 480, a USB interface 490, and a power supply unit 499 which are mounted on the main body 400M.

The control unit 410 includes a central processing unit (CPU) and an auxiliary circuit thereof, controls each part of the smartphone 400, and executes processing described below according to a program and data stored in the memory 411.

The memory 411 includes a random-access memory (RAM) used as a work area necessary for a program to be executed by the control unit 410, and a read-only memory (ROM) for storing a basic program to be executed by the control unit 410. In addition, a semiconductor memory (memory card or the like) may be used as a storage medium of an auxiliary storage device for assisting a storage area of the memory 411.

In this example, the display 420 includes a liquid crystal display (LCD) and is controlled by the control unit 410 to display predetermined images on a display screen.

In this example, the instruction unit 430 includes a touch pad (not shown) superposed on the display screen of the display 420, and inputs an instruction signal indicating an instruction by the user to the control unit 410. In this example, the display 420 and the touch pad constitute a known touch panel.

The network communication unit 480 transmits information from the control unit 410 to another device (for example, a server (not shown)) via a network 900. In addition, information from other devices is received via the network 900 and transferred to the control unit 410.

The USB interface 490 outputs information from the control unit 410, as a signal in accordance with the USB standard, to an outside, in this example, to the nebulizer head 1 through the female USB connector 491 provided on the end wall 400Me (see FIG. 1) of the main body 400M and the USB cable 200 including the USB connector 208 connected the female USB connector 491. In this example, the signal in accordance with the USB standard includes a DC voltage (DC 5 V) output through the Vbus line 201 and the GND line 204 of the USB cable 200 and a control signal (D+ signal and D− signal) having a square waveform output through the D+ line 202 and the D− line 203 of the USB cable 200. The D+ signal and the D− signal are differential signals. The Vbus line 201 can supply a current of up to 500 mA at a DC of 5 V.

In this example, the power supply unit 499 includes a rechargeable secondary battery (for example, a lithium-ion battery). The power supply unit 499 supplies a power to each part including the control unit 410, the memory 411, the display 420, the instruction unit 430, the network communication unit 480, and the USB interface 490 mounted on the main body 400M.

### (Configuration of Nebulizer Head)

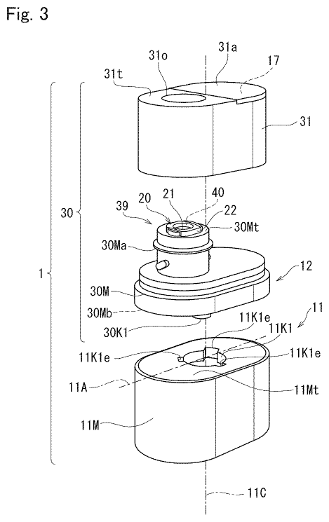

FIG. 3 shows a nebulizer head 1 in a disassembled state as viewed obliquely. The nebulizer head 1 roughly includes a main body 11 and a nebulizing unit 12 attached to the main body 11.

In this example, the main body housing 11M as a first housing forming the main body 11 has an outer shape with an oval planar shape (having a major axis 11A extending from the left front to the right back in FIG. 3) and columnarly extending in the direction of the vertical axis 11C (in this example, the vertical direction). A recess 11K1 having a substantially short cylindrical outer shape is provided in a central portion (through which the vertical axis 11C passes) of the upper wall 11Mt of the main body housing 11M as an element for detachably attaching the main body 11 and the nebulizing unit 12. In this example, the recess 11K1 includes orientation grooves 11K1e, 11K1e, and 11K1e expanded radially outward in portions corresponding to specific orientations (in this example, three orientations at intervals of 120°) around the vertical axis 11C.

The nebulizing unit 12 includes a base housing 30M having the same oval planar shape as that of the main body housing 11M, and a cover member 31 covering the base housing 30M. The cover member 31 is detachably fitted and attached to the base housing 30M in the direction of the vertical axis 11 C (in this example, from above). The base housing 30M and the cover member 31 constitute a mounting housing 30 as a second housing.

In this example, the base housing 30M includes an upper stage housing portion 30Ma protruding upward in a columnar shape at a portion eccentric to the left front side from the vertical axis 11C. The upper stage housing portion 30Ma houses a horn vibrator 40 as a vibration unit adapted to atomize a liquid (chemical liquid or the like) to be nebulized. In this example, the mesh member 20 is placed on the top surface 30Mt of the upper stage housing portion 30Ma in a state of facing the horn vibrator 40. In this example, the mesh member 20 includes a sheet 21 including a mesh portion adapted to atomize the liquid, and a flange portion 22 supporting a peripheral edge of the sheet 21. The “mesh portion” means an element that has a plurality of fine through-holes in the sheet (or the plate material) and allows a liquid to pass through these through-holes to be atomized. In this example, the mesh member 20 is configured to be disposable after one use. In this example, the horn vibrator 40 and the mesh member 20 constitute an atomizer 39.

A protrusion 30K1 having a substantially short columnar outer shape is provided in a central portion (through which the vertical axis 11C passes) of the bottom wall 30Mb of the nebulizing unit 12 as an element for detachably attaching the nebulizing unit 12 to the main body 11. In this example, the protrusion 30K1 has a shape corresponding to the recess 11K1 of the main body housing 11M. That is, the protrusion 30K1 has a substantially cylindrical shape, and includes enlarged diameter portions (not shown) protruding radially outward at portions corresponding to specific orientations (in this example, three orientations at intervals of 120°) around the vertical axis 11C. Therefore, when the nebulizing unit 12 (base housing 30M) is brought close to the main body 11 (main body housing 11M) in the direction of the vertical axis 11C (in this example, from above), the protrusion 30K1 is fitted to the recess 11K1, and the main body 11 and the nebulizing unit 12 are easily and integrally assembled. Once the main body 11 and the nebulizing unit 12 are assembled, the assembled state is maintained by a frictional force between the recess 11K1 and the protrusion 30K1. It should be noted that when the user applies a force exceeding the frictional force to separate the nebulizing unit 12 from the main body 11 in the direction of the vertical axis 11C, the nebulizing unit 12 is easily detached from the main body 11.

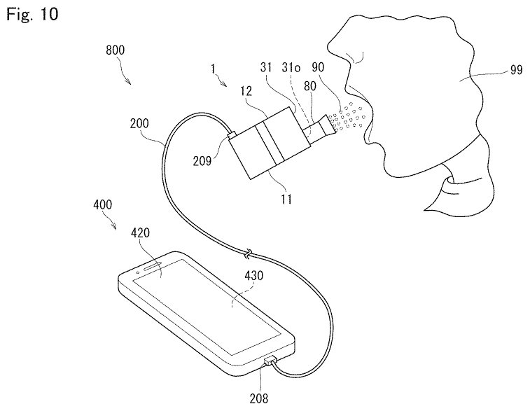

The cover member 31 has an outer shape with the same oval planar shape as that of the base housing 30M and tublarly extending in the direction of the vertical axis 11C. A circular opening 310 is provided at a portion eccentric to the left front side from the vertical axis 11C in a top wall 31t of the cover member 31. In a state where the cover member 31 is attached to the base housing 30M, the edge portion of the opening 310 presses the flange portion 22 of the mesh member 20 in the direction of the vertical axis 11C (in this example, from above). Accordingly, the sheet 21 including the mesh portion is positioned with respect to the horn vibrator 40. In addition, for example, as shown in FIG. 10, a mouthpiece 80 as a pipe member is detachably attached to the opening 310 from an outside of the cover member 31.

In addition, as shown in FIG. 3, the cover member 31 includes, at a portion corresponding to a right back side of the opening 310 in the top wall 31t, a lid portion 31a that can be opened/closed by a hinge and a liquid reservoir 17 as a liquid supply portion provided at a position immediately below the lid portion 31a. In a state where the cover member 31 is attached to the base housing 30M, the user can temporarily open the lid portion 31a and put the liquid into the liquid reservoir 17.

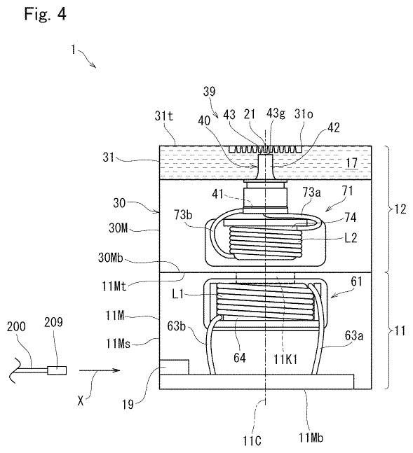

FIG. 4 schematically shows an internal structure of the nebulizer head 1 integrally assembled as viewed from a side direction. The main body 11 of the nebulizer head 1 mounts and houses a power transmission coil unit 61 and a circuit board 60 connected to the power transmission coil unit 61 by wiring lines 63a and 63b in the main body housing 11M.

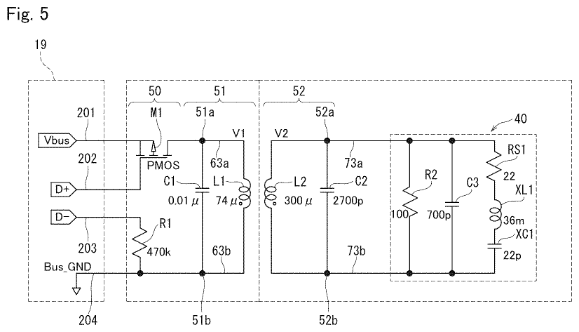

The power transmission coil unit 61 includes a pole piece 64 made of a substantially columnar magnetic body, and a power transmission coil L1 as a first coil wound and positioned around the pole piece 64. In this example, the power transmission coil unit 61 is positioned on a side facing the nebulizing unit 12 along the upper wall 11Mt of the main body housing 11M. Accordingly, the power transmission coil L1 is positioned in a region surrounding the recess 11K1 about the vertical axis 11C along an inner side of the upper wall 11Mt forming the main body housing 11M. A first capacitor C1 shown in FIG. 5 is connected in parallel to the power transmission coil L1. The power transmission coil L1 and the first capacitor C1 constitute a first resonance circuit 51. In this example, the first capacitor C1 is attached to the power transmission coil unit 61, but may be mounted on the circuit board 60.

In this example, the circuit board 60 shown in FIG. 4 is mounted with a female USB connector 19 and a conversion circuit 50 described below, and is positioned along an inner side of the bottom wall 11Mb forming the main body housing 11M. The USB connector 19 penetrates an side wall 11Ms of the main body housing 11M and opens toward the outside. A male USB connector 209 of the USB cable 200 can be inserted and connected to the USB connector 19 in a direction indicated by an arrow X in FIG. 4.

The nebulizing unit 12 mounts and houses a horn vibrator 40 and a power receiving coil unit 71 connected to the horn vibrator 40 by wiring lines 73a and 73b in the mounting housing 30 (in particular, the base housing 30M).

The horn vibrator 40 is configured by integrally combining a vibration surface 43 arranged horizontally and upwardly, an ultrasonic vibrator 41 arranged at a position spaced downwardly from the vibration surface 43, and a horn 42 arranged between the ultrasonic vibrator 41 and the vibration surface 43 to amplify the vibration of the ultrasonic vibrator 41 and transmit the vibration to the vibration surface 43. In a state where the cover member 31 is attached to the base housing 30M, a gap 43g is present between the sheet 21 including the mesh portion and the vibration surface 43 of the horn vibrator 40. As described below, the liquid in the liquid reservoir 17 is supplied to the gap 43g.

The power receiving coil unit 71 includes a pole piece 74 made of a substantially columnar magnetic body, and a power receiving coil L2 as a second coil wound and positioned around the pole piece 74. In this example, the power receiving coil unit 71 is positioned on a side facing the main body 11 along an inner side of the bottom wall 30Mb of the base housing 30M. In this assembled state, the power receiving coil L2 is electromagnetically coupled to the power transmission coil L1. A second capacitor C2 shown in FIG. 5 is connected in parallel to the power receiving coil L2. The power receiving coil L2 and the second capacitor C2 constitute a second resonance circuit 52. In this example, the second capacitor C2 is attached to the power receiving coil unit 71, but may be attached to the horn vibrator 40.

Accordingly, in a state where the main body 11 and the nebulizing unit 12 are assembled, the power transmission coil L1 and the power receiving coil L2 are arranged in regions corresponding to each other across the upper wall 11Mt forming the main body housing 11M and the bottom wall 30Mb forming the mounting housing 30. Therefore, during operation, a power for driving the horn vibrator 40 is efficiently transmitted from the power transmission coil L1 of the first resonance circuit 51 to the power receiving coil L2 of the second resonance circuit 52, in other words, from the main body 11 to the nebulizing unit 12, in this example, by a wireless power transmission system using a magnetic coupling.

As described above, since the power for driving the horn vibrator 40 is transmitted from the main body 11 to the nebulizing unit 12 by the wireless power transmission system, the main body 11 (main body housing 11M) and the nebulizing unit 12 (mounting housing 30) can be detachably separated as in this example. In a state where the main body 11 and the nebulizing unit 12 are separated from each other, for example, the base housing 30M and the cover member 31 of the nebulizing unit 12 can be washed with water separately from the main body 11.

In addition, in a state where the main body 11 and the nebulizing unit 12 are integrally assembled, there arises no disadvantage that the number of components increases and that the components are bulky. In addition, in the nebulizer head 1, the movement of the liquid from the nebulizing unit 12 (mounting housing 30) to the main body 11 (main body housing 11M) is prohibited by the outer wall (particularly, the bottom wall 30Mb of the base housing 30M) of the nebulizing unit 12. Therefore, a situation in which the liquid moves to the main body 11 and the circuit board 60 (for example, a conversion circuit 50 to be described below) fails is prevented. Furthermore, it is possible to prevent a situation in which the liquid moves from the main body 11 to the smartphone 400 through the USB cable 200 and the smartphone 400 fails.

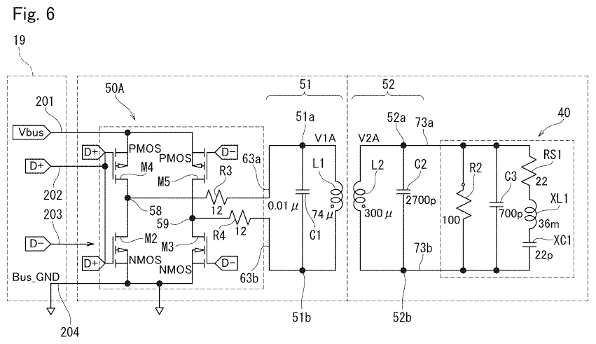

FIG. 5 illustrates the entire electric circuit included in the nebulizer head 1. The electric circuit of the nebulizer head 1 roughly includes a USB connector 19, a conversion circuit 50, a first resonance circuit 51, a second resonance circuit 52, and a horn vibrator 40 forming an atomizer 39. The electric circuit of the nebulizer head 1 does not include elements, such as a processor, a display, an instruction unit, and a battery, other than elements shown in FIG. 5 (or FIG. 6 described below).

The USB connector 19 includes a Vbus line 201, a D+ line 202, a D− line 203, and a GND line 204, correspondingly to the USB cable 200 (for simplicity, the reference signs of these lines are the same as the reference signs of the lines in the USB cable 200).

The conversion circuit 50 includes a P-channel field effect (PMOS) transistor M1 interposed between the Vbus line 201 and an one end 51a of the first resonance circuit 51, and a current limiting resistor R1 (=470 kΩ) interposed between the D− line 203 and the GND line 204. The D+ line 202 is connected to a gate of a PMOS transistor M1, and a D+ signal is applied thereto during operation. The first resonance circuit 51 includes a power transmission coil L1 (=74 μH) and a first capacitor C1 (=0.01 μF) connected in parallel to the power transmission coil L1. During operation, the PMOS transistor M1 is intermittently turned on in response to the D+ signal, and a DC voltage (Vbus=5 V) is intermittently applied between the one end 51a and the other end 51b of the first resonance circuit 51. Accordingly, the first resonance circuit 51 generates an AC voltage V1 having a sinusoidal waveform between the one end 51a and the other end 51b of the first resonance circuit 51.

The second resonance circuit 52 includes a power receiving coil L2 (=300 μH) and a second capacitor C2 (=2700 pF) connected in parallel to the power receiving coil L2. The power receiving coil L2 and the second capacitor C2 constitute a second resonance circuit 52. The power receiving coil L2 is electromagnetically coupled to the power transmission coil L1, and in this example, the coupling coefficient between the power transmission coil L1 and the power receiving coil L2 is k=0.8. In addition, the number of turns of the power receiving coil L2 is larger than the number of turns of the power transmission coil L1, and in this example, the ratio of the number of turns of the power transmission coil L1 and the number of turns of the power receiving coil L2 is set to about 1:3. Accordingly, during operation, second resonance circuit 52 increases the amplitude of the AC voltage V1 having the sinusoidal waveform generated by the first resonance circuit 51 according to the ratio between the number of turns of the power transmission coil L1 and the number of turns of the power receiving coil L2. Therefore, the second resonance circuit 52 generates an AC voltage V2 (≈3×V1) having a sinusoidal waveform between an one end 52a and the other end 52b of the second resonance circuit 52. Here, the ratio between the number of turns of the power transmission coil L1 and the number of turns of the power receiving coil L2 is set in advance so that the amplitude of the AC voltage V2 having the sinusoidal waveform with the amplitude increased (boosted) by the second resonance circuit 52 is adapted to the horn vibrator 40 forming the atomizer 39. In this example, the amplitude of the AC voltage V2 to be applied to the horn vibrator 40 is expected to be within the range of a dozen or so volts to several tens of volts.

In this example, the horn vibrator 40 forming the atomizer 39 is, as an equivalent circuit, represented by a resistance component RS1 (=22Ω), an inductive reactance component XL1 (=36 mH), and a capacitor component XC1 (=22 pF) which are connected in series, and further a capacitor component C3 (=700 pF) and a resistance component R2 (=100Ω) which are respectively connected in parallel to these entire components connected in series. During operation, the AC voltage V2 generated between the one end 52a and the other end 52b of the second resonance circuit 52 is applied to the horn vibrator 40 represented by the equivalent circuit. Accordingly, the ultrasonic vibrator 41 of the horn vibrator 40 is driven, and the vibration surface 43 shown in FIG. 4 vibrates. Then, the liquid supplied to the gap 43g between the sheet 43 including the mesh portion and the vibration surface 43 of the horn vibrator 40 is atomized through the sheet 21 including the mesh portion. In this example, since the horn vibrator 40 (the ultrasonic vibrator 41 thereof) is driven by the AC voltage V2 having the boosted sinusoidal waveform, the liquid can be efficiently atomized and ejected.

### (How to Set Frequency of AC Voltage Having Sinusoidal Waveform)

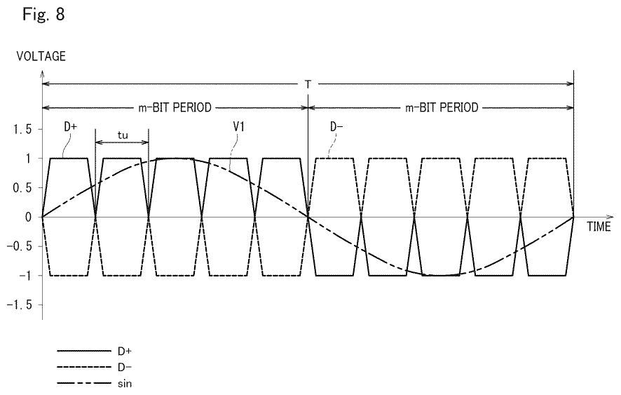

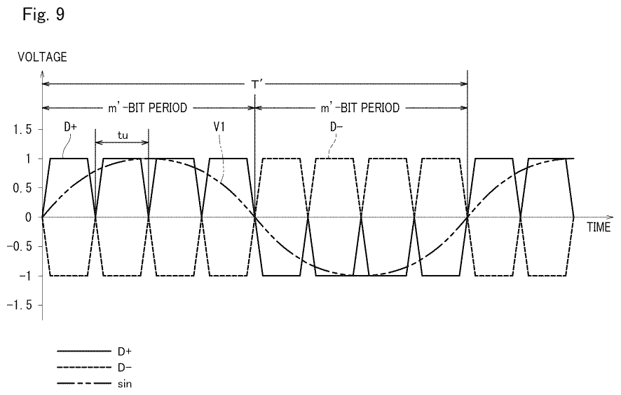

FIG. 8 shows a relationship between the control signals (D+ signal and D− signal) having the square waveform output to the nebulizer head 1 by the smartphone 400 and the AC voltage V1 having the sinusoidal waveform created using those control signals (D+ signal and D− signal) in the nebulizer head 1. In FIG. 8, the vertical axis represents a voltage, and the horizontal axis represents a time. In the vertical axis, the amplitude of the control signal and the amplitude of the AC voltage V1 are represented with normalized to 1. In the horizontal axis, the minimum period in which the level can be changed according to the transmission speed defined by the USB standard is represented as a one-bit period tu. In this example, assuming m is a natural number, the control signal is a signal repeated with a unit of 2m-bit period, in which the D+ signal (indicated by a solid line in FIG. 8) becomes at a high level (+1) for an m-bit period and then the D− signal (indicated by a broken line in FIG. 8) becomes at the high level (+1) for a following m-bit period. It should be noted that for convenience of drawing, FIG. 8 is drawn as m=5, but the present invention is not limited thereto. Since the D+ signal and the D− signal are differential signals, they change in opposite polarity. When such control signals (D+ signal and D− signal) are applied to the conversion circuit 50 shown in FIG. 5, an AC voltage V1 having a sinusoidal waveform as indicated by a two-dot chain line in FIG. 8 is generated between the one end 51a and the other end 51b of the first resonance circuit 51. That is, the AC voltage V1 becomes a positive half-wave in the m-bit period in which the D+ signal is at the high level (+1), becomes a negative half-wave in the following m-bit period in which the D− signal is at the high level (+1), and thus has a sinusoidal waveform in which the 2m-bit period (that is, a period of 2m×tu) is repeated as one period T. Therefore, the control unit 410 of the smartphone 400 can set the period T (in other words, a frequency f which is a reciprocal thereof) of the AC voltage V1 having the sinusoidal waveform by setting the 2m-bit period. In addition, the control unit 410 of the smartphone 400 can change the period T of the AC voltage V1 having the sinusoidal waveform by changing the value of m. For example, as illustrated in FIG. 9, the control unit 410 of the smartphone 400 can shorten the period T of the AC voltage V1 having the sinusoidal waveform to a 2m′-bit period (represented by T′ in FIG. 9) by reducing the value of m by 1 to m′. As described above, the frequency f of the AC voltage V1 having the sinusoidal waveform can be increased by reducing the value of m. Conversely, by increasing the value of m, the frequency f of the AC voltage V1 having the sinusoidal waveform can be lowered.

Here, examples of the transmission speed defined by the USB standard include 1.5 Mbps (USB 1.0 standard), 12 Mbps (USB 1.1 standard), and 480 Mbps (USB 2.0 standard). Since the 1-bit period tu can be shortened at a high transmission speed, the amount of change in the frequency f caused by changing the value of m by one can be reduced. Therefore, practically, a substantially continuous change (sweep) in frequency can be made as long as the frequency (for example, about 180 kHz) is for (the ultrasonic vibrator 41 of) the horn vibrator 40.

### (Operation of Nebulizer System)

It is assumed that the nebulizer head 1 is assembled in advance as shown in FIG. 4 and that the liquid to be nebulized (typically, a chemical solution) is put in the liquid reservoir 17 of the nebulizing unit 12. In addition, as shown in FIG. 10, it is assumed that the mouthpiece 80 is attached to the opening 310 of the nebulizing unit 12. As shown in FIG. 10, a user 99 who intends to use the nebulizer head 1 connects, in advance by the USB cable 200, the nebulizer head 1 and a smartphone 400 in which application software for a nebulizer is installed. Accordingly, the nebulizer system 800 is configured.

The user 99 operates the instruction unit 430 (in this example, the icon displayed on the display 420) of the smartphone 400 to start the application software for the nebulizer. Subsequently, the user instructs a start of operation of the nebulizer head 1 in the application software.

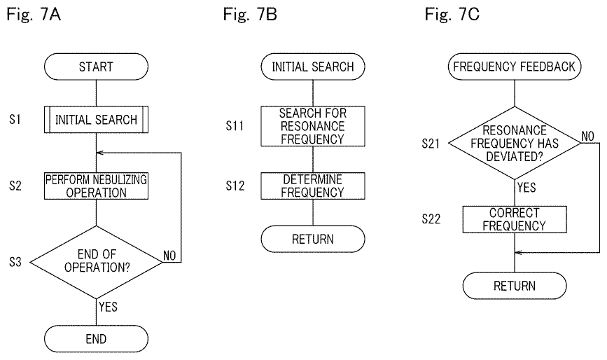

Then, as shown in step S1 in FIG. 7A, the control unit 410 of the smartphone 400 first performs the initial search processing. In this initial search processing, as shown in FIG. 7B, the control unit 410 searches for a resonance frequency by changing the frequency of the control signals (D+ signal and D− signal) output through the D+ line 202 and the D− line 203 while monitoring the current flowing between the Vbus line 201 and the GND line 204 through the USB interface 490 (step S11). Then, the control unit 410 determines the frequency f at which the largest current flows between the Vbus line 201 and the GND line 204 as the resonance frequency fr (step S12).

Subsequently, as shown in step S2 in FIG. 7A, the control unit 410 supplies a signal in accordance with the USB standard through the USB interface 490 so that the nebulizer head 1 starts a nebulizing operation at the resonance frequency fr (or near fr for stable control). Specifically, the control unit 410 supplies DC 5V through the Vbus line 201 and the GND line 204 of the USB cable 200 through the USB interface 490, and supplies control signals (D+ signal and D− signal) having the square waveform to the nebulizer head 1 through the D+ line 202 and the D− line 203 of the USB cable 200. Here, the control signal is the signal with the unit of 2m-bit period, in which the D+ signal (indicated by the solid line in FIG. 8) becomes at the high level (+1) for the m-bit period and then the D− signal (indicated by the broken line in FIG. 8) becomes at the high level (+1) for the following m-bit period. The control unit 410 sets the 2m-bit period so that the first resonance circuit 51 in the nebulizer head 1 generates an AC voltage V1 having a sinusoidal waveform at the resonance frequency fr (or near fr). Specifically, the value of m is set so that 2m×tu≈1/fr. In this manner, the control unit 410 supplies the signal according to the USB standard in which the value of m is set to the nebulizer head 1 through the USB interface 490.

Then, in the nebulizer head 1, the conversion circuit 50 shown in FIG. 5 intermittently applies DC 5 V to the first resonance circuit 51 (including the power transmission coil L1 and the first capacitor C1) according to the control signals (D+ signal and D− signal) to generate the AC voltage V1 having the sinusoidal waveform at the resonance frequency fr (or near fr). The AC voltage V1 having the sinusoidal waveform generated by the first resonance circuit 51 is boosted by the second resonance circuit 52 (including the power receiving coil L2 and the second capacitor C2) to become an AC voltage V2 having the sinusoidal waveform. The horn vibrator 40 of the atomizer 39 shown in FIG. 4 is driven by the AC voltage V2 having this sinusoidal waveform, and the vibration surface 43 vibrates. Accordingly, the liquid supplied to the gap 43g between the sheet 21 including the mesh portion and the vibration surface 43 of the horn vibrator 40 is atomized through the sheet 21 including the mesh portion, and is ejected as an aerosol 90 through the mouthpiece 80 as shown in FIG. 10.

When the nebulizing operation is continued, the current resonance frequency fr of the horn vibrator 40 may deviate from the initial resonance frequency (this is referred to as fr0) due to various causes such as a temperature change. Therefore, the control unit 410 of the smartphone 400 executes a frequency feedback processing shown in FIG. 7C. In the frequency feedback processing, as shown in step S21, the control unit 410 determines whether or not the frequency f of the AC voltage V1 having the sinusoidal waveform generated by the first resonance circuit 51 in the nebulizer head 1 has deviated from the initial resonance frequency fr0 by monitoring the current flowing between the Vbus line 201 and the GND line 204 through the USB interface 490. Then, if there has occurred a frequency deviation (YES in step S21), the control unit 410 corrects the frequency f so that the frequency f of the AC voltage V1 having the sinusoidal waveform generated by the first resonance circuit 51 in the nebulizer head 1 matches the current resonance frequency fr of the horn vibrator 40 based on the current flowing between the Vbus line 201 and the GND line 204 (step S22). The frequency feedback processing is repeatedly executed as needed during the nebulizing operation. That is, the control unit 410 determines whether or not the frequency f of the AC voltage V1 having the sinusoidal waveform generated by the first resonance circuit 51 in the nebulizer head 1 has deviated from an immediately preceding resonance frequency fr0. Then, if there has occurred a frequency deviation (YES in step S21), the control unit 410 corrects the frequency f so that the frequency f of the AC voltage V1 having the sinusoidal waveform generated by the first resonance circuit 51 in the nebulizer head 1 matches an current resonance frequency fr of the horn vibrator 40 (step S22).

In this example, the nebulizing operation is continued as long as an end of operation of the nebulizer head 1 is not instructed (NO in step S3 in FIG. 7A. When the user 99 operates the instruction unit 430 (in this example, the icon displayed on the display 420) of the smartphone 400 to instruct an end of operation of the nebulizer head 1 (YES in step S3 in FIG. 7A), the control unit 410 stops the supply of the signal according to the USB standard to the nebulizer head 1. Accordingly, the nebulizer head 1 ends the nebulizing operation.

It should be noted that timer setting may be enabled on the application software for the nebulizer of the smartphone 400, and the nebulizing operation may be automatically ended when a predetermined nebulizing operation time is completed.

In this way, according to this nebulizer system 800, the user 99 can perform a nebulizing operation using the nebulizer head 1. In this nebulizer system 800, no controller (including a processor) is interposed between the smartphone 400 and the nebulizer head 1, and the nebulizer head 1 also does not include a processor. Therefore, the nebulizer system 800 can be configured compactly with a small number of components.

In addition, in the nebulizer head 1, since the atomizer 39 is of a mesh type, the nebulizer head 1 is configured to be relatively small and can be driven with a relatively low power. Therefore, it is suitable to miniaturize the nebulizer head 1 and to configure the nebulizer system 800 compactly.

### Modification

In the above example, in the electric circuit shown in FIG. 5, during operation, the PMOS transistor M1 forming the conversion circuit 50 is intermittently turned on according to the D+ signal, and the DC voltage (Vbus=5 V) is intermittently applied between the one end 51a and the other end 51b of the first resonance circuit 51. However, the present invention is not limited thereto, and as in an electric circuit shown in FIG. 6, a DC voltage (Vbus=5 V) may be alternately polarity-reversed to be applied between the one end 51a and the other end 51b of the first resonance circuit 51 during operation.

In an example in FIG. 6, an H-bridge type conversion circuit 50 A is provided instead of the conversion circuit 50. In the conversion circuit 50A, between the Vbus line 201 and the GND line 204, a PMOS transistor M4 and an N-channel field effect (NMOS) transistor M2 are connected in series in this order between the Vbus line 201 and the GND line 204, and a PMOS transistor M5 and an NMOS transistor M3 are connected in series in this order, respectively. A connection point 58 between the PMOS transistor M4 and the NMOS transistor M2 is connected to the one end 51a of the first resonance circuit 51 through a current-limiting resistor R3 (=12Ω). A connection point 59 between the PMOS transistor M5 and the NMOS transistor M3 is connected to the other end 51b of the first resonance circuit 51 through a current-limiting resistor R4 (=12Ω). A D+ line 202 is connected to each of a gate of the PMOS transistor M4 and a gate of the NMOS transistor M2, and a D+ signal is applied thereto during operation. In addition, a D− line 203 is connected to each of a gate of the PMOS transistor M5 and a gate of the NMOS transistor M3, and a D− signal is applied thereto during operation.

With this configuration, during operation, the PMOS transistor M4 and the NMOS transistor M3 at diagonal positions (upper left and lower right) in FIG. 6 are alternately turned on and off in the same phase according to the D+ signal and the D− signal, respectively. In addition, in an opposite phase to the phase, the NMOS transistor M2 and the PMOS transistor M5 at the other diagonal positions (lower left and upper right) in FIG. 6 are alternately turned on and off in the same phase according to the D+ signal and the D− signal, respectively. As a result, a DC voltage (Vbus=5 V) is alternately polarity-reversed and applied between the one end 51a and the other end 51b of the first resonance circuit 51. Accordingly, the first resonance circuit 51 generates an AC voltage VIA having a sinusoidal waveform between the one end 51a and the other end 51b of the first resonance circuit 51. The amplitude of the AC voltage VIA is about twice the amplitude of the AC voltage V1 shown in FIG. 5. Accordingly, the amplitude of the AC voltage V2A having a sinusoidal waveform generated by the second resonance circuit 52 is also about twice the amplitude of the AC voltage V2 shown in FIG. 5. Therefore, the present invention can be preferably adapted to when the horn vibrator 40 forming the atomizer 39 is of a type (specification) that needs to be driven at a relatively high voltage.

In the above-described embodiment, the system of transmitting the power for driving the horn vibrator 40 from the main body 11 to the nebulizing unit 12 in the nebulizer head 1 is the wireless power transmission system. However, the present invention is not limited thereto. A system of transmitting power for driving the horn vibrator 40 from the main body 11 to the nebulizing unit 12 may be a wired power transmission system. In a case of the wired power transmission system, for example, the main body housing 11M of the main body 11 and the base housing 30M of the nebulizing unit 12 may be made to an integral housing in the nebulizer head 1, and the AC voltage V1 having the sinusoidal waveform generated by the conversion circuit 50 shown in FIG. 5 or the AC voltage VIA having the sinusoidal waveform generated by the conversion circuit 50A shown in FIG. 6 may be applied to the horn vibrator 40 by wiring. In this case, the housing of the nebulizer head 1 can be simplified, and the configuration of the electric circuit of the nebulizer head 1 can be simplified. Therefore, the nebulizer head 1 can be configured compactly with a smaller number of components.

In addition, in the above embodiment, the electric circuit of the nebulizer head 1 includes no element other than the elements shown in FIG. 5 or FIG. 6. However, the present invention is not limited thereto. For example, the circuit board 60 of the nebulizer head 1 may mount an integrated circuit (IC) having a function of returning information to the smartphone 400, in which the information may be that USB communication between the smartphone 400 and the nebulizer head 1 has been established. In addition, a display such as a light emitting diode (LED) that displays a signal supply state from the smartphone 400 to the nebulizer head 1 may be provided on the side surface 11Ms (in particular, a portion visible from the user 99 shown in FIG. 10) of the main body housing 11M of the nebulizer head 1. For example, it is desirable that the display is turned on to indicate that the signal is being supplied during a period in which a signal (in particular, the control signal) according to the USB standard is supplied from the smartphone 400 to the nebulizer head 1, and is turned off to indicate that the signal supply is stopped during other periods.

In addition, in the above embodiment, (the circuit board 60 of) the main body 11 is mounted with the female USB connector 19, and the male USB connector 209 of the USB cable 200 is connected to the USB connector 19. However, the present invention is not limited thereto. One end of the USB cable 200 may be directly attached to (the conversion circuit 50 or 50A of) the circuit board 60 by soldering or the like without interposing the USB connectors 19 and 209, and the USB cable 200 may directly protrude from the main body 11 (the main body housing 11M) (USB cable direct outlet structure).

In addition, in the above embodiment, the nebulizer head 1 (the main body 11 and the nebulizing unit 12) has an oval planar shape, but the present invention is not limited thereto. The planar shape of the nebulizer head 1 may be an ellipse, a circle, a rounded quadrangle (quadrangle rounded at corners), or the like.

In addition, in the above-described embodiment, the nebulizer system 800 includes the smartphone 400 as a computer device capable of supplying a signal according to the USB standard through the USB cable, but the present invention is not limited thereto. Instead of the smartphone 400, for example, a personal digital assistant (PDA), a tablet terminal, a personal computer, or the like may be provided.

As described above, a nebulizer system of the present disclosure is a nebulizer system for atomizing and ejecting a liquid, the nebulizer system comprising:
a computer device configured to supply a signal according to a USB standard through a USB cable,
wherein the computer device supplies a DC voltage through a Vbus line and a GND line of the USB cable, and supplies a control signal having a square waveform through a D+ line and a D− line of the USB cable; and
a nebulizer head directly connected to the computer device by the USB cable,
wherein the nebulizer head includes:
a first resonance circuit including a first coil and a first capacitor,
a conversion circuit that applies the DC voltage intermittently or alternately polarity-reversedly according to the control signal to the first resonance circuit to generate an AC voltage having a sinusoidal waveform, and
an atomizer that is driven by the AC voltage having the sinusoidal waveform and atomizes and ejects the liquid.

In the present specification, the “USB cable” is intended to include a cable capable of transmitting a signal according to the USB standard and a connector (this is referred to as a “USB connector”) provided at an end portion of the cable.

The “control signal” refers to a D+ signal and a D− signal, which are differential signals.

The “directly connected” by the USB cable means that a connection is made between the computer device and the nebulizer head without any intervening of a controller (including a processor) in the conventional example. It should be noted that it is not excluded that the computer device and/or the nebulizer head comprise(s) a USB connector, to which the USB connector at the end portion of the USB cable can be connected.

In the nebulizer system of the present disclosure, a DC voltage is directly supplied from the computer device to the nebulizer head through the Vbus line and the GND line of the USB cable, and a control signal having a square waveform is supplied through the D+ line and the D− line of the USB cable. In the nebulizer head, the conversion circuit applies the DC voltage to the first resonance circuit (including a first coil and a first capacitor) intermittently or alternately polarity-reversedly according to the control signal to generate an AC voltage having a sinusoidal waveform. The atomizer is driven by the AC voltage having the sinusoidal waveform, and atomizes and ejects the liquid. Therefore, the nebulizer system can atomize and eject the liquid without interposing a controller (including a processor) between the computer device and the nebulizer head. In addition, the nebulizer head also does not need to include a processor. Therefore, the nebulizer system can be configured compactly with a small number of components.

In the nebulizer system of one embodiment,
the nebulizer head further includes a second resonance circuit including a second coil electromagnetically coupled to the first coil and a second capacitor, and
a power for driving the atomizer is transmitted from the first resonance circuit to the second resonance circuit by a wireless power transmission system.

In the nebulizer system according to this one embodiment, since the power for driving the atomizer is transmitted from the first resonance circuit to the second resonance circuit by the wireless power transmission system, the housing of the nebulizer head can be configured to be detachably separated, for example, between the first coil and the second coil.

In the nebulizer system of one embodiment,
the nebulizer head includes, detachably from each other,
a first housing on which the first resonance circuit and the conversion circuit are mounted, and
a second housing on which the second resonance circuit and the atomizer are mounted, and
a movement of the liquid from the second housing to the first housing is prohibited by an outer wall of the second housing.

In the nebulizer system of this one embodiment, in the nebulizer head, the first housing and the second housing are detachable from each other. In a state where the first housing and the second housing are separated, the second housing can be washed with water separately from the first housing. In a state where the first housing and the second housing are integrally assembled, there arises no disadvantage that the number of components increases and that the components are bulky. In addition, in the nebulizer head, a movement of the liquid from the second housing to the first housing is prohibited by the outer wall of the second housing. Therefore, it is possible to prevent a situation in which the liquid moves to the first housing and the conversion circuit fails. Furthermore, it is possible to prevent a situation in which the liquid moves from the first housing to the computer device through the USB cable and the computer device fails.

In the nebulizer system of one embodiment,
a number of turns of the second coil is larger than a number of turns of the first coil,
the second resonance circuit increases an amplitude of the AC voltage having the sinusoidal waveform generated by the first resonance circuit according to a ratio between the number of turns of the first coil and the number of turns of the second coil, and
the atomizer is driven by the AC voltage having the sinusoidal waveform with the amplitude increased by the second resonance circuit.

In the nebulizer system of this one embodiment, the second resonance circuit increases the amplitude of the AC voltage having the sinusoidal waveform generated by the first resonance circuit according to the ratio between the number of turns of the first coil and the number of turns of the second coil. The atomizer is driven by an AC voltage having the sinusoidal waveform with the amplitude increased by the second resonance circuit, and atomizes and ejects the liquid. Therefore, by setting in advance the ratio between the number of turns of the first coil and the number of turns of the second coil so that the amplitude of the AC voltage having the sinusoidal waveform with the amplitude increased (boosted) by the second resonance circuit is adapted to the atomizer, the liquid can be efficiently atomized and ejected.

In the nebulizer system of one embodiment,
the atomizer includes:
a horn vibrator having a vibration surface, the horn vibrator operating using the AC voltage having the sinusoidal waveform, and
a mesh member having a mesh portion positioned facing the vibration surface, and
the atomizer atomizes the liquid supplied between the vibration surface and the mesh portion through the mesh portion during operation.

In the nebulizer system of this one embodiment, the atomizer atomizes the liquid supplied between the vibration surface and the mesh portion through the mesh portion during operation. Such a mesh-type atomizer is configured to be relatively small and can be driven with a relatively low power. Therefore, it is suitable to miniaturize the nebulizer head and to configure the nebulizer system compactly.

In the nebulizer system of one embodiment,
assuming that a 1-bit period is a minimum period in which a D+ signal and a D− signal constituting the control signal can change in level according to a transmission speed defined by the USB standard, and m is a natural number, the control signal is a signal repeated with a unit of 2m-bit period, in which the D+ signal becomes at a high level for an m-bit period and then the D− signal is becomes at the high level for a following m-bit period.

Examples of the transmission speed defined by the USB standard include 1.5 Mbps (USB 1.0 standard), 12 Mbps (USB 1.1 standard), and 480 Mbps (USB 2.0 standard).

As described above, the conversion circuit applies the DC voltage to the first resonance circuit (including the first coil and the first capacitor) intermittently or alternately polarity-reversedly according to the control signal to generate the AC voltage having the sinusoidal waveform. Here, in the nebulizer system of this one embodiment, the control signal is repeated with a unit of 2m-bit period, in which the D+ signal becomes at a high level for an m-bit period and then the D− signal is becomes at the high level for a following m-bit period. Therefore, the period of the AC voltage having the sinusoidal waveform, which the conversion circuit causes the first resonance circuit to generate, is the 2m-bit period. Therefore, the computer device can set the period (in other words, a frequency which is a reciprocal thereof) of the AC voltage having the sinusoidal waveform by setting the 2m-bit period.

In the nebulizer system of one embodiment, the computer device changes a period of the AC voltage by changing a value of the m of the control signal.

In the nebulizer system of this one embodiment, the computer device can easily change the period (in other words, the frequency that is the reciprocal thereof) of the AC voltage having the sinusoidal waveform by changing the value of the m.

As is apparent from the above, the nebulizer system of the present disclosure can be configured compactly with a small number of components.

The above embodiments are illustrative, and are modifiable in a variety of ways without departing from the scope of this invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. A nebulizer system for atomizing and ejecting a liquid, the nebulizer system comprising:
a computer device configured to supply a signal according to a USB standard through a USB cable,
wherein the computer device supplies a DC voltage through a Vbus line and a GND line of the USB cable, and supplies a control signal having a square waveform through a D+ line and a D− line of the USB cable; and
a nebulizer head directly connected to the computer device by the USB cable,
wherein the nebulizer head includes:
a first resonance circuit including a first coil and a first capacitor,
a conversion circuit that applies the DC voltage intermittently or alternately polarity-reversedly according to the control signal to the first resonance circuit to generate an AC voltage having a sinusoidal waveform, and
an atomizer that is driven by the AC voltage having the sinusoidal waveform and atomizes and ejects the liquid.

2. The nebulizer system according to claim 1, wherein
the nebulizer head further includes a second resonance circuit including a second coil electromagnetically coupled to the first coil and a second capacitor, and
a power for driving the atomizer is transmitted from the first resonance circuit to the second resonance circuit by a wireless power transmission system.

3. The nebulizer system according to claim 2, wherein
the nebulizer head includes, detachably from each other,
a first housing on which the first resonance circuit and the conversion circuit are mounted, and
a second housing on which the second resonance circuit and the atomizer are mounted, and
a movement of the liquid from the second housing to the first housing is prohibited by an outer wall of the second housing.

4. The nebulizer system according to claim 2, wherein
a number of turns of the second coil is larger than a number of turns of the first coil,
the second resonance circuit increases an amplitude of the AC voltage having the sinusoidal waveform generated by the first resonance circuit according to a ratio between the number of turns of the first coil and the number of turns of the second coil, and
the atomizer is driven by the AC voltage having the sinusoidal waveform with the amplitude increased by the second resonance circuit.

5. The nebulizer system according to claim 1, wherein
the atomizer includes:
a horn vibrator having a vibration surface, the horn vibrator operating using the AC voltage having the sinusoidal waveform, and
a mesh member having a mesh portion positioned facing the vibration surface, and
the atomizer atomizes the liquid supplied between the vibration surface and the mesh portion through the mesh portion during operation.

6. The nebulizer system according to claim 1, wherein
assuming that a 1-bit period is a minimum period in which a D+ signal and a D− signal constituting the control signal can change in level according to a transmission speed defined by the USB standard, and m is a natural number, the control signal is a signal repeated with a unit of 2m-bit period, in which the D+ signal becomes at a high level for an m-bit period and then the D− signal is becomes at the high level for a following m-bit period.

7. The nebulizer system according to claim 6, wherein the computer device changes a period of the AC voltage by changing a value of the m of the control signal.
